(19) United States
(12) Patent Application Publication
(10) Pub. No.: US 2023/0390374 A1
(43) Pub. Date: Dec. 7, 2023

(54) MULTI-ANTIGEN BACTERIAL OUTER MEMBRANE VESICLE AND USE THEREOF

(71) Applicant: BIOMVIS SRL, Siena (SI) (IT)

(72) Inventors: Assunta Gagliardi, Lagonegro (PZ) (IT); Alberto Grandi, SIENA (IT); Laura Fantappie', Firenze (FI) (IT)

(73) Assignee: BIOMVIS SRL, Siena (SI) (IT)

(21) Appl. No.: 18/031,993

(22) PCT Filed: Oct. 19, 2021
(86) PCT No.: PCT/EP2021/078940

§ 371 Date: Apr. 14, 2023

(30) Foreign Application Priority Data
Oct. 19, 2020 (EP) 20202631.6

(51) Int. Cl. A61K 39/085; C07K 14/31; A61P 31/04

(52) U.S. Cl. CPC A61K 39/085; C07K 14/31; A61P 31/04; A61K 2039/70

(57) ABSTRACT

There is disclosed a method for the co-expression of multi-antigens in bacterial outer membrane vesicles, immunogenic compositions containing the isolated vesicles and the use thereof for the prevention or treatment of bacterial infections.

The invention relates to a method for multi-antigen co-expression in bacterial outer membrane vesicles (OMVs) and to immunogenic compositions containing the isolated vesicles. The method of invention is conveniently applied to the development of vaccines useful for the prevention or treatment of bacterial infections.

### BACKGROUND

Bacterial Outer Membrane Vesicles (OMVs)

All Gram negative bacteria spontaneously release outer membrane vesicles (OMVs) during growth both in vitro and in vivo. OMVs are closed spheroid particles, 20-300 nm in diameter, generated through a “budding out” of the bacterial outer membrane. Consistent with that, the majority of OMV components are represented by LPS, glycerophospholipids, outer membrane proteins, lipoproteins and periplasmic proteins (Kulp A and Kuehn M J (2010) Annu. Rev. Microbiol. 64, 163-184; Ellis T N and Kuehn M J (2010) Microbiol. Mol. Biol. Rev. 74, 81-94).

OMVs represent a distinct secretory pathway with a multitude of functions, including inter and intra species cell-to-cell cross-talk, biofilm formation, genetic transformation, defense against host immune responses and toxin and virulence factor delivery to host cells (Kulp A and Kuehn M J (2010) Annu. Rev. Microbiol. 64, 163-184). OMVs interaction to host cells can occur by endocytosis after binding to host cell receptors or lipid rafts. Alternatively, OMVs have been reported to fuse to host cell membrane, leading to the direct release of their content into the cytoplasm of the host cells (Kulp A and Kuehn M J (2010) Annu. Rev. Microbiol. 64, 163-184; Ellis T N and Kuehn M J (2010) Microbiol. Mol. Biol. Rev. 74, 81-94).

OMVs purified from several pathogens, including Neisseria, Salmonella, Pseudomonas, Vibrio cholerae, Burkholderia, and E. coli, induce potent protective immune responses against the pathogens they derive from (Collins B S (2011) Discovery Medicine, 12, 7-15), and highly efficacious anti-Neisseria OMV-based vaccines are already available for human use (Hoist J et al. (2009) Vaccine, 27S, B3-B12). Such remarkable protection is attributed to two main properties of OMVs. First, they carry the proper immunogenic and protective antigens which, in extracellular pathogens, usually reside on the surface and therefore are naturally incorporated in OMVs. Indeed, OMV immunization induces potent antibody responses against the major membrane-associated antigens. However, OMV immunogenicity is not restricted to antibody responses. For instance, mice immunized with Salmonella OMVs develop robust Salmonella-specific B and T cell responses, and OMVs stimulate IFN-γ production by a large proportion of CD4+ T cells from mice previously infected with Salmonella, indicating that OMVs are an abundant source of antigens recognized by Salmonella-specific CD4+ T cells (Alaniz R C et al. (2007) J. Immunol. 179, 7692-7701). Second, OMVs possess a strong “built-in” adjuvanticity since they carry many of the bacterial Pathogen-Associated-Molecular Patterns (PAMPs) which, by binding to pathogen recognition receptors (PRRs), play a key role in stimulating innate immunity and in promoting adaptive immune responses. OMV-associated PAMPs include LPS which, in concert with MD-2 and CD14, binds TLR-4, lipoproteins whose acylpeptide derivatives interact with TLR-1/2 and 2/6 heterodimers, and peptidoglycan whose degradation products bind to intracellular NOD1/2 (Moshiri A et al. (2012) Hum. Vaccines. Immunother 8, 953-955; Ellis T N et al., (2010) Inn. Immun. 78, 3822-3831; Kaparakis M et al., (2010) Cell. Miocrobiol. 12, 372-385). The engagement of this group of PPRs results in the activation of transcription factors (NF-kB) and the consequent expression of specific cytokines. Interestingly, LPS, lipoproteins and peptidoglycan can work synergistically, thus potentiating the built-in adjuvanticity of OMVs (Chen D J et al., (2010) Proc Natl Acad Sci USA, 107, 3099-3104).

OMVs also have the capacity to induce protection at the mucosal level. Protection at the mucosal sites is known to be at least partially mediated by the presence of pathogen-specific IgAs and Th17 cells. In particular, a growing body of evidence suggests that Th17 cells have evolved to mediate protective immunity against a variety of pathogens at different mucosal sites. Interestingly, Th17 cells have recently also been shown to play a crucial role in the generation of vaccine-induced protective responses. For instance, it has been reported that in mice whole cell pertussis vaccines (Pw) induce Th17 cells and neutralization of IL-17 after vaccination reduces protection against a pulmonary challenge with B. pertussis. Similarly, in a CD4+ T cell dependent, antibody-independent model of vaccine-induced protection following S. pneumoniae challenge, treatment with anti-IL-17 antibodies resulted in reduced immunity to pneumococcal colonization compared to the control serum treated mice (Malley R et al (2006) Infect Immun., 74, 2187-2195). Elicitation of IgAs and Th17 cells by OMVs has been well documented and this can explain mechanistically the good protective activities of OMVs against several mucosal pathogens. For instance, immunization with Vibrio cholerae-derived OMVs protects rabbits against Vibrio cholerae oral challenge (Roy N et al (2010) Immunol. Clinical Microbiol. 60, 18-27) and Pasteurella multocida-derived and Mannheimia haemolytica-derived OMVs protect mice from oral challenge with P. multocida (Roier S et al (2013) Int. J. Med. Microbiol. 303, 247-256). In addition, intranasal immunization with Porphyromonas gingivalis OMVs elicits potent IgA production at both serum and mucosal level and immunization with Escherichia coli—derived OMVs prevent bacteria-induced lethality. Protective effect of Escherichia coli—derived OMVs is primarily mediated by OMV-specific, IFN-γ and IL-17 producing, T cells (Kim O Y et al (2013) J. Immunol. 190, 4092-4102).

In addition to their “built-in” adjuvanticity, OMVs are becoming a promising vaccine platform for two main reasons: (1) the ease with which they can be produced in large scale, and (2) possibility to be engineered with heterologous proteins.

OMV production—In general, the amount of OMVs released by Gram-negative bacteria when grown under laboratory conditions is too low to allow their exploitation in biotechnological applications. However, two approaches can be used to enhance the yields of OMVs and make them compatible with industrial applications. One approach exploits the addition of mild detergents to the bacterial biomass to promote the vesiculation process and, at the same time, to decrease the level of OMV reactogenicity by removing a substantial amount of LPS (Fredriksen J H et al (1991) NIPH Ann. 14, 67-79). Although this process has been proved to produce safe and effective vaccines against Meningococcal B (Granoff D. (2010), Clin. Infect. Dis. 50, S54-S65; Crum-Cianflone N. and Sullivan E. (2016) Infect Dis Ther., 5, 89-112) its main drawback is that the detergent treatment favours bacterial cell lysis with the consequence that the OMV preparations are heavily contaminated with cytoplasmic proteins (Ferrari G. et al (2006) Proteomics, 6, 1856-1866). The second approach to enhance OMV production is to insert into the genome of the OMV-producing strain mutations that enhance vesiculation. For instance, in Neisseria meningitidis, a mutation in the gna33 gene, encoding a glucosyltransferase, has been shown to drive the release of several milligrams of vesicles per liter in the culture supernatant (Ferrari G et al (2006) Proteomics, 6, 1856-1866). Similar quantities of vesicles are obtained from Escherichia coli strains carrying deletions in the genes encoding the Tol/Pal system (a protein complex involved in the connection of the inner membrane with the outer membrane) (Bernadac A. et al (1998) J. Bacteriol. 180, 4872-4878) and in the ompA gene, encoding one of the major outer membrane proteins of E. coli (Fantappiè L. et al. (2014) J. Extracellular Vesicles, 3, 24015). Such quantities make the production process of OMVs highly efficient and inexpensive. A number of other mutations have been described that enhance the production of OMVs in several Gram-negative bacteria, including Salmonella and E. coli (Deatherage B L et al (2009) Mol. Microbiol. 72, 1395-1407; McBroom A J and Kuehen M J (2007) Mol. Microbiol. 63, 545-558; Kulp A et al (2015) PLos ONE 10, e0139200). Recently, it was shown that the cumulative inactivation of genes encoding periplasmic and membrane-associated proteins leads to the isolation of E. coli strains featuring a hyper-vesiculating phenotype (PCT/EP2020/060762).

As far as the purification of OMVs from the culture supernatant is concerned, centrifugation and tangential flow filtration (TFF) are commonly used. The yield of OMV production using centrifugation couple to TFF can easily exceed 100 mg/liter of culture (Berlanda Scorza F. et al (2012) PLos ONE 7, e35616) and therefore the process is perfectly compatible with large scale production.

OMV engineering—OMVs can be manipulated in their protein content by genetic engineering. This feature was demonstrated for the first time by Kesty and Kuehn who showed that Yersinia enterocolitica outer membrane protein Ail assembled on OMVs surface when expressed in E. coli, and that the GFP fluorescence protein fused to the “twin arginine transport (Tat)” signal sequence was incorporated in the OMV lumen (Kesty N C and Kuhen M J (2004) J. Biol. Chem. 279, 2069-2076). Following the observation by Kesty and Kuehn, an increasing number of heterologous proteins have been successfully delivered to OMVs using a variety of strategies. For instance, heterologous antigens have been delivered to the surface of OMVs by fusing them to the β-barrel forming autotransporter AIDA and to hemolysin ClyA, two proteins that naturally compartmentalized into E. coli OMVs (Schroeder J and Aebischer T (2009) Vaccine, 27, 6748-6754; Chen D J et al (2010) Proc. Natl. Acad. Sci. USA, 107, 3099-3104). Heterologous antigens from Group A Streptococcus and Group B Streptococcus were delivered to the lumen of E. coli vesicles by fusing their coding sequences to the leader peptide of E. coli OmpA (Fantappie L et al (2014) J. Extracellular Vesicles 3, 24015). Recently, five different S. aureus antigens were delivered in the membrane of OMVs by fusing their coding sequence to the leader peptide of E. coli Lpp, demonstrating that different strategies can be successfully used to drive the expression of a large variety of antigens in the OMVs compartment. The level of expression of the five antigens in OMVs was from 5 to 20% of total OMV proteins. This level was consistently superior to what observed when the same proteins were delivered to the luminal compartment of the vesicles by expressing them in the periplasm (Irene C et al (2019) Proc. Natl. Acad. Sci. USA, 116: 21780-21788). Interestingly, when the recombinant vesicles were used to immunize mice, they elicited high titers of functional antibodies against the heterologous antigens and showed to confer protection to infection by S. pyogenes and S. aureus in different mouse challenge models.

Many vaccines against infectious diseases require more than one antigen to be effective. The need of multi-component vaccines is dictated by a number of reasons, including the variability of the clinical isolates and the virulence mechanisms of the pathogens, which often produce several virulence factors which have to be neutralized by specific immune responses. For instance, several serotypes of Streptococcus pneumoniae infect humans, each differing for the polysaccharide capsule they produce. To provide sufficiently broad protection, the current anti-S. pneumoniae vaccine includes 13 glycoconjugates, synthesized using the purified capsules of the most common serotypes. Both Bordetella pertussis and Neisseria meningitidis group B vaccines are constituted by five pathogen-specific protein antigens. Only such combinations, and not the single antigens, promote the elicitation of antigen-specific immune responses, which synergize and confer immunity against the two pathogens. If the inclusion of more than one antigen is often indispensable to elicit protective immune responses, it poses a challenge from a production standpoint. A number of production processes equal to the number of antigens present in the vaccine has to be set-up and ultimately each purified component has to be combined with the others to make the final formulation. Such process has a relevant impact on the overall process and vaccine costs.

The same drawback described above holds for OMV-based, multi-component vaccines. Although the production and the purification of OMVs decorated with single antigens is simple and inexpensive, OMV-based, multi-component vaccines still require the purification of as many engineered OMVs as the number of antigens needed in the vaccine.

Vaccines against S. aureus

S. aureus is a commensal Gram-positive bacterium in humans and animals but is responsible for severe diseases when it becomes invasive. This usually occurs in patients with immunological or barrier defects, but highly pathogenic strains have recently emerged that have the ability to cause diseases in otherwise healthy individuals (Tong S Y C et al (2015) Clin. Microbiol. Rev. 28(3), 603-661.).

Colonization is the key risk factor for S. aureus community and hospital-acquired invasive diseases. In the USA, approximately 3.4 million community-acquired diseases and 340.000 hospital acquired-diseases occur annually, leading to more than 30.000 deaths (DeLeo F R and Chambers H F (2009) J. Clin. Invest. 19(9), 2464-2474).

One of the most serious problems with S. aureus is its antibiotic resistance. A growing number of clinical isolates are being described that, largely through horizontal transfer, have acquired genetic traits which make them resistant to most antibiotics (Foster T J (2017) FEMS Microbiol Rev. 41(3), 430-449). Suffice to say that in less than three decades from its introduction, approximately 80% of all isolates were reported to be penicillin-resistant. The most effective way to solve the problem of antibiotic resistance would be vaccination.

However, despite the several decades of intense research by numerous world-class laboratories, an anti-S. aureus vaccine is still far from being available. An explanation for the absence of an effective vaccine can be found in the biology and pathogenesis of S. aureus. The invasive strains are characterized by the expression of a myriad of virulent factors and of more than 35 secreted evasion molecules which make S. aureus the champion of pathogens in circumventing the defense mechanisms of the mammalian immune system (Foster T J (2005) Nat Rev Microbiol. 12, 948-958; Liu G Y (2009) Pediatr Res 65, 1R-77R). Moreover, once phagocytosed by professional immune cells, S. aureus has the ability to escape the killing mechanisms and phagocytes can become the vehicles with which the pathogen disseminates itself inside the host, reaching out and infecting vital organs (Thammavongsa V et al (2015) Nat Rev Microbiol. 13(9), 529-543). Therefore, the traditional strategies that have been exploited for the development of many effective anti-bacterial vaccines, strategies which are largely based on the elicitation of antibodies inactivating toxins/virulent factors and/or promoting bactericidal/opsonophagocytic activity, are probably not applicable for such a sophisticated and ingenious pathogen.

Three Phase III S. aureus vaccine trials have been reported: the two component CP5/CP8 glycoconjugate vaccine (Fattom A et al (2015) Hum. Vaccin. Immunother. 11(3), 632-41), the single component IsdB vaccine (Fowler V G et al (2013) JAMA 309, 1368-1378.) and the four component CP5/CP8 glycoconjugates/ClfA/MntC vaccine (Anderson A S et al (2012) Hum. Vaccin. Immunother., 8(11), 1585-1594). However, the efficacy data of the three vaccines, all formulated without adjuvants, were largely disappointing.

The failures of all three vaccine trials can be attributed to (1) inappropriate antigen selection in terms of both quality and quantity, and (2) adjuvant selection.

Antigen Selection

In consideration of the redundancy of virulent factors expressed by the different S. aureus isolates it is surely a difficult task to take the decision on which antigens are necessary and sufficient to elicit a broad protective immunity. As already pointed out, S. aureus produces a plethora of virulence factors and toxins, which have been shown to play important roles in pathogenesis. Many of them have been tested as vaccine candidates in different animal models with promising results (Bagnoli F, Rappuoli R, Grandi, G (Eds.) (2017) Staphylococcus aureus: Microbiology, Pathology, Immunology, Therapy and Prophylaxis, Current Topics in Microbiology and Immunology, Springer). They include secreted toxins such as the pore-forming leukocidins, the S. aureus supernatigens and super-antigen-like toxins, the lipoproteins and the LPXTG-anchored proteins.

Adjuvant Selection

A second, nonexclusive explanation of the vaccine failures described above is that the traditional strategies used to develop anti-bacterial vaccines, strategies largely based on the elicitation of neutralizing and/or bactericidal antibodies, are not adequate for this pathogen. As recently pointed out (Irene C et al. (2019) Proc Natl Acad Sci USA. 116(43), 21780-21788), the ability of S. aureus to survive inside phagocytic cells might require a paradigm shift in vaccine design and administration. In particular, eliciting Th1/Th17-skewed adaptive immune responses acting in concert with a strong T cell-independent innate-type of immunity would be of great importance to enhance the killing capacity of phagocytic cells. A number of adjuvants have been developed to elicit Th1/Th17 skewed immune responses, some of them are already part of vaccine formulations for human use, while others are being tested in clinical trials (Lamine Mbow M. (2010) Curr Opin Immunol 22(3), 411-6). OMVs appear to be particularly attractive for a S. aureus vaccine. They carry components capable of stimulating different pathways of innate immunity and they promote strong T-independent immune responses.

### DISCLOSURE OF THE INVENTION

The present inventors have surprisingly found that the OMVs can be engineered to express a plurality of fusion proteins, each fusion protein consisting of a plurality of bacterial antigens, and that such engineered OMVs provide a valuable means for generating an immune response against multiple antigens and thereby conferring a broad protection against infections caused by one or more bacterial species.

According to a first aspect the invention provides a method of producing a bacterial outer membrane vesicle (OMV), which comprises:
(i) providing a plurality of polynucleotides, wherein each polynucleotide encodes a fusion product containing (a) at least two different bacterial proteins or (poly)peptides capable of eliciting an immune response in a host, optionally separated by a peptide linker, and (b) a leader sequence for secretion at the 5′ end, wherein said polynucleotides preferably differ from each other for at least one, more preferably at least two and most preferably for all of the encoded proteins or (poly)peptides contained in the fusion product;
(ii) inserting the polynucleotides in expression plasmids, one plasmid for each different polynucleotide, thereby obtaining a plurality of plasmids;
(iii) introducing the plurality of plasmids in a Gram-negative bacterium;
(iv) growing the bacterium under suitable conditions to produce the OMVs.

The bacterial proteins or (poly)peptides capable of eliciting an immune response in a host can be either heterologous antigens which are not produced by the Gram-negative bacterium from which the OMVs according to the invention are isolated, or antigens naturally expressed in the OMV-producing bacterial strain.

The bacterial proteins or (poly)peptides used in the method of invention can be from the same or different bacterial species and preferably they are bacterial antigens known to be involved in the infection process e.g. as virulence factors.

Bacterial species suitable as source of antigens for use according to the invention include, but are not limited to:
Neisseria meningitidis: useful antigens include membrane proteins such as adhesins, autotransporters, toxins, iron acquisition proteins, factor H binding protein (fHbp), Neisseria Heparin-Binding Antigen (NHBA), NadA;
Streptococcus pneumoniae: useful polypeptide antigens include the RrgB pilus subunit, the beta-N-acetyl-hexosaminidase precursor, serine/threonine kinase StkP and pneumococcal surface adhesin PsaA;
Streptococcus pyogenes: useful antigens include Slo, SpyCEP and Spy 0269 proteins;
Streptococcus agalactiae (Group B Streptococcus): useful antigens include the pilus proteins belonging to the three different pilus islands (Margarit I et al., J Infect Dis. (2009); 199(1): 108-15);
Moraxella catarrhalis;
Bordetella pertussis: Useful pertussis antigens include acellular or whole-cell pertussis antigens, pertussis holotoxin or toxoid (PT), filamentous haemagglutinin (FHA), pertactin, and agglutinogens 2 and 3;
Pseudomonas aeruginosa;
Chlamydia trachomatis: Useful antigens include PepA, LcrE, ArtJ, DnaK, OmpH-like, L7/L12, OmcA, AtoS, CT547, Eno, HtrA and MurG and HtrA;
Helicobacter pylori: Useful antigens include, but are not limited to, CagA, VacA, NAP, and/or urease;
Escherichia coli: Useful antigens include, but are not limited to, antigens derived from enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC) and/or enterohemorrhagic E. coli (EHEC). ExPEC strains include uropathogenic E. coli (UPEC) and meningitis/sepsis-associated E. coli (MNEC);
Francisella, such as F. novicida, F. philomiragia, F. tularensis;
Neisseria gonorrhoeae;
Treponema pallidum;
Haemophilus ducreyi;
Enterococcus faecalis or Enterococcus faecium;
Staphylococcus saprophyticus;
Yersinia enterocolitica;
Mycobacterium tuberculosis;
Mycobacterium leprae;
Rickettsia;
Listeria monocytogenes;
Vibrio cholerae;
Salmonella typhi;
Borrelia burgdorferi;
Porphyromonas gingivalis;
Klebsiella;
Rickettsia prowazekii;
Legionella pneumophila.

In a preferred embodiment, the antigens are derived from Staphylococcus aureus and they are selected from the group consisting of: IsdB, Fe-scavenging, cell wall-anchored protein (Stranger-Jones Y K et al (2006) Proc Natl Acad Sci USA. n103(45), 16942-16947; Harro C et al (2010) Clin Vaccine Immunol 121868-1874); IsdA, Iron-regulated surface determinant protein A (Stranger-Jones Y K et al (2006) Proc Natl Acad Sci USA. n103(45), 16942-16947); SdrD Serine-aspartate repeat-containing protein D (Stranger-Jones Y K et al (2006) Proc Natl Acad Sci USA. n103(45), 16942-16947); SdrE Serine-aspartate repeat-containing protein E (Stranger-Jones Y K et al (2006) Proc Natl Acad Sci USA. n103(45), 16942-16947); ClfB (Clumping factor B) (Schaffer A C et al (2006) Infect Immun. 74(4), 2145-2153); FnBP fibronectin-binding protein (Zhou H et al, (2006) Vaccine. 24(22), 4830-4837); FhuD2 ferric hydroxamate uptake D2 (Bagnoli F et al (2015) Proc Natl Acad Sci USA, 112(12), 3680-3685); conserved staphylococcal antigen 1A (Bagnoli F et al (2015) Proc Natl Acad Sci USA 112(12), 3680-3685); EsxA ess extracellular A and EsxB ess extracellular B (Bagnoli F et al (2015) Proc Natl Acad Sci USA, 112(12), 3680-3685); LukSPV Panton Valentine Leukocidin S (Spaan A N et al (2017) Nat Rev Microbiol. 435-447); TSST-1 toxic shock syndrome toxin-1 (Llewelyn M AND Cohen J (2002) Lancet Infect Dis 2(3), 156-162); HlgAB gamma-hemolysin AB (Spaan A N et al (2017) Nat Rev Microbiol. 15(7), 435-447); HlgCB gamma-hemolysin CB (Spaan A N et al (2017) Nat Rev Microbiol. 15(7), 435-447); LukED Leukocidin ED (Spaan A N et al (2017) Nat Rev Microbiol. 15(7), 435-447); LukAB Leukocidin AB (Spaan A N et al (2017) Nat Rev Microbiol. 15(7), 435-447); SEs A B, C, D, E, G, H, I, R, T Enterotoxins (Llewelyn M AND Cohen J (2002) Lancet Infect Dis 2(3), 156-62.); SEls J, K, L, M, N, O, P, Q, S, U, V, X Enterotoxins like (Llewelyn M AND Cohen J (2002) Lancet Infect Dis. 2(3), 156-162); PBP2a Penicillin Binding Protein 2A (Haghighat M et al (2017) Mol Immunol. 91, 1-7); ETA, ETB Exfoliative toxins (Gillet G et al (1997) Clin Infect Dis, 25(6), 1369-1373); SAR0280, EsaA and Member of the Type VII secretion system EsaA family; SAR0735, Hypothetical protein; SAR0992, Serine protease; SAR1262, Ribonuclease Y; SAR1402, Phosphate-binding protein; SAR1489, Cell surface elastin binding protein; SAR1507, Phage tail tape measure protein; SAR1795, Septation ring formation regulator EzrA; SAR2104, Hypothetical protein; SAR2496, Zinc ABC transporter substrate-binding protein; SAR2635, O-acetyltransferase (cell wall biosynthesis); SAR2723, N-acetylmuramoyl-L-alanine amidase; SAR2753, Lipase (Rasmussen K J et al. (2016) Vaccine. 34, 4602-4609).

In a more preferred embodiment, said S. aureus antigens are selected from Protein A “SpA” (encoding sequence: SEQ ID NO:1; amino acid sequence: SEQ ID NO:2) and particularly SpA_{KKAA} (encoding sequence: SEQ ID NO:3, amino acid sequence: SEQ ID NO:4), Clumping Factor A “ClfA” (encoding sequence: SEQ ID NO:5, amino acid sequence: SEQ ID NO:6) and particularly ClfA_{Y338A} (encoding sequence: SEQ ID NO:7, amino acid sequence: SEQ ID NO:8), α-hemolysin “Hla” (encoding sequence: SEQ ID NO:9, amino acid sequence: SEQ ID NO:10) and particularly Hla_{H35L} (encoding sequence: SEQ ID NO:11, amino acid sequence: SEQ ID NO:12), Leukocidin-subunit E “LukE” (encoding sequence: SEQ ID NO:13, amino acid sequence: SEQ ID NO:14), or variants thereof having at least 40%, preferably at least 60%, more preferably at least 80% sequence identity.

Protein A (SpA) binds immunoglobulins (Ig) and Ig binding contributes quite substantially to S. aureus virulence and toxicity. This occurs through the inhibition of the opsonophagocytosis. Moreover, SpA acts as a potent B cell super-antigen triggering the secretion of all VH3 antibodies, irrespectively of their antigen specificity. Finally, SpA is responsible for an anaphylactic syndrome due to the SpA binding to the VH3 region of IgG and IgE antibodies associated to basophils and mast cells. It was shown that immunization with the SpA_{KKAA}, a mutant no longer capable of binding Fcγ and VH3, induced antibodies inhibiting Ig binding, promoting S. aureus Newman and MRSA USA300 LAC OPK in mouse, guinea pig and human blood (Kim H K et al (2012) Infect Immun. 80(10), 3460-3470. Kim H K et al (2015) mBio. 6(1), e02369-14;) and protecting animals from bacteraemia (Kim H K et al (2010) J Exp Med. 207(9), 1863-1870).

Clumping Factor A (ClfA) is a surface-exposed virulence factor expressed in most S. aureus isolates and whose primary function is to allow the adhesion of S. aureus to fibrinogen (McDevitt D et al., (1997) Eur J Biochem. 247, 416-424; Cheng A G et al., (2009) FASEB J. 23, 3393-3404; Josefsson E et al., (2008) PLoS One. 3:e2206; Hawkins J et al., (2012) Clin Vaccine Immunol. 19, 1641-1650; Scully I L et al., (2015) Vaccine 33, 5452-5457). Inhibiting the fibrinogen binding capacity of ClfA has been proposed as a way to prevent pathogenesis. Indeed, immunization of monkeys and human volunteers with recombinant ClfA, particularly with the N1N2N3 portion of the protein carrying the Y338A mutation, which abolishes the ClfA binding to fibrinogen (Josefsson E et al. (2008) PLoS One. 3:e2206), elicited high titers of functional antibodies (Hawkins J et al., (2012) Clin Vaccine Immunol. 19, 1641-1650).

S. aureus α-hemolysin (Hla—also known as α-toxin) is the founding member of a family of bacterial pore-forming β-barrel toxins (Bhakdi S and Tranum-Jensen J (1991) Microbiol. Rev. 55, 733-751; Song L et al (1996) Science. 274, 1859-1866), which assembles in a heptameric structure forming a pore of 2-nm in diameter into the plasma membrane (Gouaux E et al (1997) Protein Sci. 6, 2631-2635). Hla is expressed by almost all clinical isolates (Berube B J and Bubeck Wardenburg J (2013) Toxins 5, 1140-66.) Passive transfer of monoclonal antibodies against Hla were shown to be highly protective in different animal models of S. aureus infection (Trang T T V et al. (2020) Antimicrob Agents Chemother. 64(3): e02220-19; Rouha H et al. (2015) MAbs. 7(1), 243-254) and anti-Hla mAbs are being tested in different clinical trials. HlaH35L, a nontoxigenic variant of Hla carrying a mutation that prevents pore formation (Menzies B E and Kernodle D S (1994) Infect. Immun. 62, 1843-1847), has been extensively tested as a vaccine candidate. HlaH35L was shown to protect mice against pneumonia (Bubeck Wardenburg J and Schneewind O (2008) J Exp Med. 205(2), 287-94), lethal sepsis and kidney abscess (Rauch S et al. (2012) Infect Immun. 80(10), 3721-3732.) and skin infection (Bagnoli F et al. (2015) Proc Natl Acad Sci USA. 112(12), 3680-3685.) and a HlaH35L containing vaccine is current in clinical trials (Bagnoli F, Rappuoli R, Grandi, G (Eds.) (2017) Staphylococcus aureus: Microbiology, Pathology, Immunology, Therapy and Prophylaxis, Current Topics in Microbiology and Immunology, Springer). In addition to Hla, human S. aureus isolates produce five pore-forming toxins known as leukocidins (Spaan A N et al. (2017) Nat Rev Microbiol. 15(7), 435-447). Leukocidins are constituted by two subunits, the host cell targeting S component and the polymerization F component. The major target of leukotoxins are immune cells of myeloid and lymphoid lineages, suggesting that these toxins have evolved to inhibit both the innate and adaptive immunity. Among the five leukocidins, LukED appears to be particularly important in that it kills virtually all immune cells, including neutrophils, monocytes, macrophages, dendritic cells, T cells, erythrocytes and NK cells (Spaan A N et al. (2017) Nat Rev Microbiol. 15(7), 435-447). The toxin is encoded in the stable S. aureus pathogenicity island vSaf353, which is present in about 70% of all clinical isolates (Baba T et al. (2009) J Bacteriol. 191, 1180-1190). Because of their key role in pathogenesis, inactivated leukocidins are considered to be key targets for both therapeutic and prophylactic intervention (Spaan A N et al. (2017) Nat Rev Microbiol. 15(7), 435-447).

In one embodiment, the fusion product contains 2, 3, or 4, preferably 2, different bacterial proteins or (poly)peptides. The protein or (poly)peptide sequences can be variously arranged within the fusion product, i.e. with different reciprocal orientation of their N- and C-termini.

In a preferred embodiment, the fusion product contains 2 different bacterial antigens A and B such that its encoding polynucleotide is a DNA construct of the formula LS-Antigen A-Linker-Antigen B, wherein “LS” is a leader sequence for secretion.

In a more preferred embodiment, the fusion product is selected from the following groups (a) and (b), wherein the proteins can be fused to each other with or without interposition of a linker:
(a) Spa fused to Hla, preferably SpA_{KKAA} fused to Hla_{H35L} (SEQ ID NO:15)
(b) ClfA fused to LukE, preferably ClfA_{Y338A} fused to LukE (SEQ ID NO:16).

According to the invention, the leader sequence for secretion is a leader sequence which promotes the translocation of the fusion proteins into the periplasm of the Gram-negative bacterium, thus allowing the compartmentalization of the fusion proteins in the lumen of the OMV. Alternatively, the leader sequence for secretion is a lipoprotein leader sequence which promotes the translocation of the fusion proteins into the outer membrane of the Gram-negative bacterium thus allowing the compartmentalization of the fusion proteins as lipidated proteins in the membrane of the OMV. When anchored to the membrane, the lipidated fusion proteins can protrude out to the OMV surface. An example of a leader sequence that can be used in the present invention is the signal sequence of the murein lipoprotein Lpp (MKATKLVLGAVILGSTLLAGCSS—SEQ ID NO:19). However, any other suitable lipoprotein signal sequence can be used.

According to the invention, the term “linker” designates any peptide sequence containing from 1 to 20 amino acids and preferably 2 to 10 amino acids. Although the linker may contain any amino acid, the residues which are preferably mostly represented in the peptide linker are Glycine (Gly) and Serine (Ser).

Different fusion proteins can be expressed in the same OMV by cloning the encoding nucleotide sequences in different expression plasmids carrying compatible origin of replication. The term “compatible origin of replication” means that different plasmids can replicate in the same bacterial cell. More precisely, different plasmids with compatible origins of replication can be stably inherited together by bacterial cell also in the absence of external selection (Novick R. P. et al (1976) Bacteriol Rev. 40(1): 168-189).

The expression plasmids capable of replication in a Gram-negative bacterium are preferably selected from pGEX, pUC19, pALTR, pQE, pLEX, pHAT.

The most common dual-plasmid pair is based on plasmids having ColE1 (or pMB1) and p15A as origins of replication. The most common plasmid triplet is based on plasmids having ColE1 (or pMB1), p15A, and pSC101 as origins of replication.

In one embodiment, the Gram-negative bacterium used in the method of invention is E. coli. The bacterium is cultured in conditions suitable for growth and vesiculation, which include the use of rich media such as LB supplemented with additional carbon and nitrogen sources, or chemically defined media using different carbohydrates as carbon sources. Growth temperatures typically vary from 20° C. to 37° C. and the supernatants containing the vesicles can be collected toward the end of the exponential phase or in the stationary phase of growth, depending upon the growth conditions in use. The conditions suitable for bacterial growth and vesiculation are known to anyone skilled in the art and are described for instance in Berlanda Scorza, F. et al. “High yield production process for Shigella outer membrane particles”, PLoS One 7, e35616 (2012).

The OMVs of the invention can be obtained from any suitable Gram-negative bacterium. The Gram-negative bacterium is typically E. coli, but any Gram-negative bacterium that is not pathogenic in humans can be used as well. For example, the bacteria may be commensalistic in humans or they are not typically found in human hosts at all. Exemplary species for use in the invention include species in any of genera Escherichia, Shigella, Neisseria, Moraxella, Bordetella, Borrelia, Brucella, Chlamydia Haemophilus, Legionella, Pseudomonas, Yersinia, Helicobacter, Salmonella, Vibrio, etc. In particular, the bacterium may be a Shigella species (such as S. dysenteriae, S. flexneri, S. boydii or S. sonnei). Alternatively, it may be a Neisseria species, particularly a non-pathogenic species such as N. bacilliformis, N. cinerea, N. elongata, N. flavescens, N. lactamica, N. macacae, N. mucosa, N. polysaccharea, N. sicca or N. subflava, and in particular N. lactamica. Alternatively, a pathogenic species of Neisseria may be used, e.g. N. gonorrhoeae or N. meningitidis. In other examples, the bacterium may be Bordetella pertussis, Borrelia burgdorferi, Brucella melitensis, Brucella ovis, Chlamydia psittaci, Chlamydia trachomatis, Moraxella catarrhalis, Haemophilus influenzae (including non-typeable stains), Legionella pneumophila, Pseudomonas aeruginosa, Yersinia enterocolitica, Helicobacter pylori, Salmonella enterica (including serovars typhi and typhimurium, as well as serovars paratyphi and enteritidis), Vibrio cholerae, Proteus, Citrobacter, Serratia, Envinia, Pasteurella etc. Photosynthetic Gram-negative bacteria may also be used. Typically, the bacterium is a competent strain. This feature facilitates genetic modification of the bacterium.

In a further aspect, the invention provides an isolated bacterial outer membrane vesicle (OMV) carrying in the lumen or in the membrane a plurality of bacterial proteins or (poly)peptides capable of eliciting an immune response in a host, wherein said proteins or (poly)peptides are fused to each other in groups of two or more, optionally separated by a peptide linker.

In a preferred embodiment, four bacterial proteins or (poly)peptides are fused to each other in groups of two, optionally with the interposition of a peptide linker, whereby two fusion products are generated.

In a particularly preferred embodiment, the bacterial proteins or (poly)peptides are antigens from Staphylococcus aureus.

In a particularly preferred embodiment, such S. aureus antigens are selected from Protein A (SpA), Clumping Factor A (ClfA), α-hemolysin (Hla), Leukocidin E (LukE), which are fused to one another in groups of two. Particularly preferred is an isolated OMV carrying the following fusion products in the membrane or in the lumen:
(a) Spa fused to Hla, preferably SpA_{KKAA} fused to Hla_{H35L}
(b) ClfA fused to LukE, preferably LS-ClfA_{Y338A} fused to LukE.

A further aspect of the invention relates to an immunogenic composition containing an OMV carrying a plurality of bacterial proteins or (poly)peptides in the lumen or in the membrane, as herein disclosed.

The composition of the invention is in a suitable administration form and it is preferably in the form of a vaccine. Vaccines according to the invention may either be prophylactic or therapeutic. Pharmaceutical compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age and the capacity of the individual's immune system. The amount of OMVs in the compositions of the invention may generally be between 10 and 500 μg, preferably between 25 and 200 μg, and more preferably about 50 μg or about 100 μg.

Compositions of the invention may be prepared in various liquid forms. For example, the compositions may be prepared as injectables, either as solutions or suspensions. The composition may be prepared for pulmonary administration e.g. by an inhaler, using a fine spray. The composition may be prepared for nasal, aural or ocular administration e.g. as spray or drops, and intranasal vesicle vaccines, as known in the art. Other suitable administration routes include intramuscular, oral, parenteral, transmucosal, or intradermal administration. Injection may be via a needle (e.g. a hypodermic needle), but needle-free injection may alternatively be used.

A further aspect of the invention relates to the isolated OMV or the immunogenic composition thereof, as herein disclosed, for use in the stimulation of an immune response in a subject in need thereof. Preferably, said subject is an individual with a disease correlated to or caused by a bacterial infection or an individual at risk of developing a bacterial infection. In a preferred embodiment, such bacterial infection is caused by S. aureus.

### DESCRIPTION OF THE FIGURES

FIG. 1. Cloning strategy used to clone different chimeric-multi antigens in different expression plasmids. The two gene fusions, clfA_{Y338A}-lukE and spA_{KKAA}-hla_{H35L}, were chemically synthesized. They have been designed to contain at the 5′ end the leader sequence of the lipoprotein Lpp (Lipobox: Leu-(Ala/Ser)-(Gly-Ala)-Cys) for expression as lipoproteins fused to the coding region of the first gene. The second gene is fused to the first and separated by a short linker. Once synthetized, the DNA strings can be amplified by PCR with specific forward and reverse primers and then cloned in both pET and pACYC plasmids, two compatible vectors that differ in copy number. Gene expression was driven by an inducible T7 promoter. The resulting plasmids pET-ClfA_{Y338A}-LukE and pACYC-SpA_{KKAA}-Hla_{H35L} were used to co-transform the E. coli BL21(DE3)Δ60 strain.

FIG. 2. SDS-PAGE and WB analysis of OMVs from E. coli BL21(DE3)Δ60_pET(ClfA_{Y338A}-LukE)/pACYC(SpA_{KKAA}-Hla_{H35L}) strain, expressing heterologous antigens. (A)E. coli BL21(DE3)Δ60 strain expressing the lipidated chimeric antigens was grown in lysogeny (Luria-Bertani) broth (LB) at 37° C. and after 2 hours of induction with IPTG at 30° C. OMVs were purified from culture supernatants by tangential flow filtration (TFF). Aliquots (20m of total OMV proteins) were analyzed by SDS-PAGE. Twenty μg of Empty OMVs obtained from E. coli BL21(DE3)Δ60 strain were also loaded as negative control (B) The presence of chimeric antigens in CLSH-OMVs was confirmed by Western blot. Five μg of empty OMVs and of CLSH-OMVs were loaded onto a 4-12% SDS-polyacrylamide gel and then the separated proteins were transferred to nitrocellulose filters. As primary antibodies, anti-HlaH35L, anti-LukE or anti-Spa_{KKAA} rabbit polyclonal antibodies, obtained by immunizing rabbits with specific synthetic peptides, or anti-ClfA_{Y338A} mouse polyclonal antibodies obtained by immunizing mouse with domain N1N2N3 of rClfA_{Y338A} (aa 40-559) were used.

FIG. 3. Analysis of antigen lipidation by Triton X-114 fractionation of OMV proteins. CLSH-OMVs were dissolved by adding 1% Triton X-114 at 4° C. and subsequently aqueous and detergent phases were partitioned by centrifugation. Unfractionated total proteins from OMVs (Total), hydrophilic proteins in the aqueous phase (AQ phase), and hydrophobic proteins in the detergent phase (DT phase) were precipitated with chloroform/methanol and separated by SDS-PAGE. Finally, proteins were transferred onto nitrocellulose filters and the presence of the chimeric antigens in the detergent phases was checked by Western blot using antigen-specific antibodies.

FIG. 4. Analysis of antigen-specific IgG titers induced in mice immunized with CLSH-OMVs. CD1 mice were immunized three times at 2-wk interval with CLSH-OMVs formulated with 2 mg/ml alum hydroxide. Sera were collected 7 days after the third immunization and IgG titers were analyzed by ELISA using plates coated with recombinant proteins LukE, SpA_{KKAA}, HlaH35L and N1N2N3 rClfA_{Y338A} (0.3 μg/well).

FIG. 5. Analysis of functional immune responses in mice immunized with CLSH-OMVs. (A) Functional activity of anti-Hla_{H35L} antibodies elicited by CLSH-OMVs. Twenty ng of rHla were incubated with rabbit erythrocytes in the presence or absence of different dilutions of sera from mice immunized with Alum alone, Empty OMVs or CLSH-OMVS in Alum. Inhibition of Hla hemolytic activity is given as percentage of hemolytic activity obtained incubating the rabbit erythrocytes with water (100% hemolysis). (B) Functional activity of anti-ClfA antibodies elicited by CLSH-OMVs. Different dilutions of sera from mice immunized with Empty OMVs (grey bars) or CLSH-OMVs (black bars), formulated in Alum, were pre-incubated with 10\hat{ }7 CFUs of S. aureus Newman strain. Then bacteria plus immune sera were transferred to a 96-well plate previously coated with human fibrinogen (Fg) (10 μg/ml). Supernatant was removed, adherent bacteria were fixed with formaldehyde and then stained with crystal violet. Percentage of inhibition of bacterial binding to Fg was calculated with respect to maximal bacterial binding (bacteria without serum, 100% binding).

FIG. 6. In vivo protective activity of CLSH-OMVs in sepsis, skin and kidney abscess models of infection of mice with S. aureus Newman strain. (A) Sepsis model of infection. Groups of 8 CD1 female mice were immunized three times at 2-wk interval with Alum alone, Empty OMVs or CLSH-OMVs, formulated in alum. After 2 wk, mice were infected intraperitoneally with a lethal dose of S. aureus Newman strain (3×10\hat{ }8 CFUs). Protection data are reported as % survival at day 7. Statistical analysis was performed using Student's T-test (***P<0.0001). (B) Skin model of infection. Groups of 8 CD1 female mice were immunized three times at 2-wk interval with Alum alone (empty square), Empty OMVs (filled circle) or CLSH-OMVs (filled diamonds), formulated in alum. At 14 d after the third immunization, mice were s.c. infected with 5×10\hat{ }7 CFUs of S. aureus Newman strain. Abscess size was monitored once per day for 12 d. Results indicate the mean±SEM for all groups. (C) Renal abscess model. Groups of 7 CD1 female mice were immunized three times at 2-wk interval with Alum alone (circles), Empty OMVs (triangles) or CLSH-OMVs (squares), formulated in alum. Ten days after the last immunization, mice were infected i.v. with a sublethal dose of S. aureus Newman strain (1×10\hat{ }7 CFUs) and 4 d afterward, mice were sacrificed, kidneys collected and homogenized in PBS, and finally aliquots were plated on agar media for CFU determination. Bars indicate the geometric mean±95% CI for each group. Statistical analysis was performed using Student's T-test (*P<0.05).

### DETAILED DESCRIPTION OF THE INVENTION

Cloning of Two Protein Chimeras in Two Different Plasmids

Two gene fusions were generated, clfA_{Y338A}-lukE and spA_{KKAA}-hla_{H35L}. ClfA_{Y338A}-LukE chimeric protein is composed of the N1N2N3 (aa 40-559) domain of ClfA_{Y338A} fused to the LukE protein. SpA_{KKAA}-HlaH35L chimeric protein is composed of the Ig binding region of Staphylococcal protein A_{KKAA} (SpA_{KKAA}) fused to Hla_{H35L}. Each fusion carries a lipoprotein leader sequence at the 5′ end for expression as lipoproteins in the outer membrane compartment of E. coli. Different lipoprotein leader sequences can be used, for example the Lpp one. Lpp is an endogenous E. coli lipoprotein which carries a signal peptide characterized by the specific conserved sequence Leu-(Ala/Ser)-(Gly/Ala)-Cys (SEQ ID NO:23) at its C-terminal region in which the cysteine residue is lipidated.

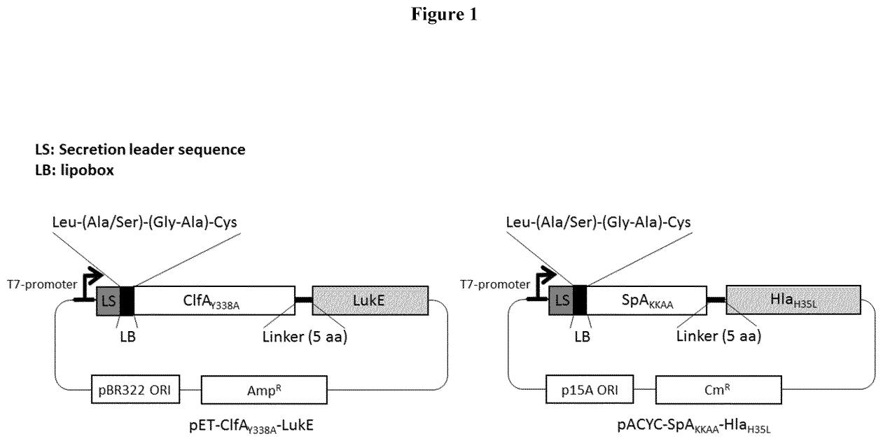

The gene fusions were cloned in both pET and pACYC, two compatible plasmids which differ in copy number. Different strategies can be used to fuse two genes together and clone them as a chimeric protein in a vector for expression. Such procedures are well known to those skills in the art. One possibility is, for example, to chemically synthetize a DNA fragment which contains the lipoprotein leader sequence and the two selected genes fused together and spaced by a short linker, as depicted in FIG. 1 (clfA_{Y338A}-lukE, nucleotide and amino acid sequences SEQ ID NOs: 17 and 18, respectively; spA_{KKAA}-hla_{H35L}, nucleotide and amino acid sequences SEQ ID NOs:15 and 16, respectively). Once synthetized the DNA string can be amplified by PCR with specific forward and reverse primers and cloned in the selected vector.

The resulting plasmids pET-ClfA_{Y338A}-LukE/pACYC-SpA_{KKAA}-Hla_{H35L} were then used to transform the hyper-vesiculating strain E. coli BL21(DE3)Δ60 (disclosed in PCT/EP2020/060762, WO2020/212524).

Analysis of Chimeric Antigens Expression

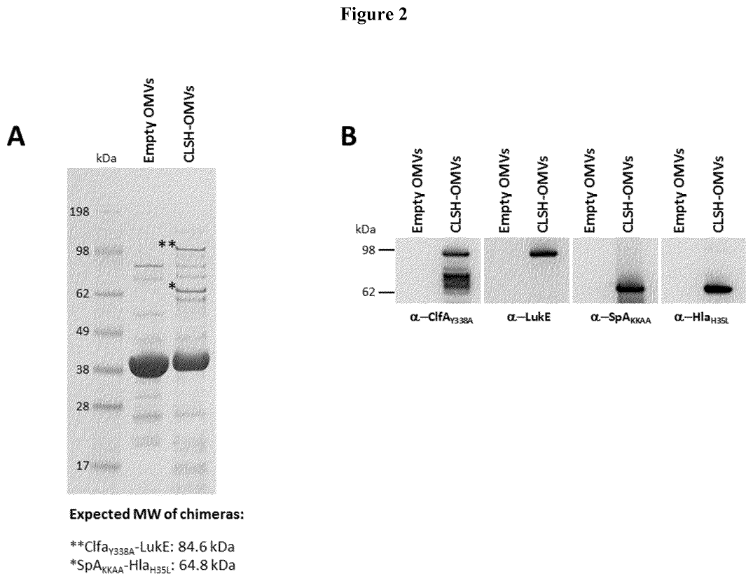

To investigate if the lipidated fusion proteins were expressed in the E. coli BL21(DE3)Δ60 strain and could be incorporated into the OMVs, the strain was grown in LB medium supplemented with ampicillin and chloramphenicol and when cultures reached an OD₆₀₀ value of 0.5, IPTG was added at 0.1 mM final concentration. After two additional hours of growth at 30° C., vesicles were purified from culture supernatants by tangential flow filtration (TFF). The presence of the fusions in the OMV preparation was analyzed by SDS-PAGE. As shown in FIG. 2A, both chimeras accumulated in the vesicular compartment at a level between 5.5% to 16.8% of total OMV proteins, as determined by densitometry scanning. The resulting OMVs carrying the two fusion proteins were named CLSH-OMVs.

To confirm the presence of the two fusion proteins antigens in the OMVs compartment a Western blot using the antibodies against the four selected antigens was also performed. In essence, 5 μg of empty OMVs and CLSH-OMVs were loaded onto a 4-12% SDS-polyacrylamide gels and then the separated proteins were transferred to nitrocellulose filters. The filters were blocked overnight at 4° C. by agitation in blocking solution (10% skimmed milk and 0.05% Tween in PBS), followed by incubation for 90 minutes at 37° C. with a 1:1.000 dilution of anti-Hla, anti-LukE or anti-SpA rabbit polyclonal antibodies, obtained by immunizing rabbits with specific synthetic peptides (CGTNTKDKWIDRSSE (SEQ ID NO:20) for Hla, CNEFVTPDGKKSAHD (SEQ ID NO:21) for LukE, CAKKLNDAQAPKADN (SEQ ID NO:22) for SpA) conjugated with Keyhole Limpet Hemocyanin (KLH) protein, or anti-ClfA mouse polyclonal antibody obtained by immunizing mice with domain N1N2N3 of rClfA_{Y338A} (aa 40-559) in 3% skimmed milk and 0.05% Tween in PBS. After 3 washing steps in PBS-Tween, the filters were incubated in a 1:2.000 dilution of peroxidase-conjugated immunoglobulin (Dako) in 3% skimmed milk and 0.05% Tween in PBS for 1 hour, and after 3 washing steps, bound conjugated IgGs were detected using the Super Signal West Pico chemo-luminescent substrate (Pierce).

As shown in FIG. 2B the two chimeric proteins are co-expressed and compartmentalized in the OMVs purified from the E. coli BL21(DE3)Δ60-pET(ClfA_{Y338A}-LukE)/pACYC(SpA_{KKAA}-Hla_{H35L}) strain.

Analysis of Lipidation of Heterologous Chimeric Antigens in OMVs

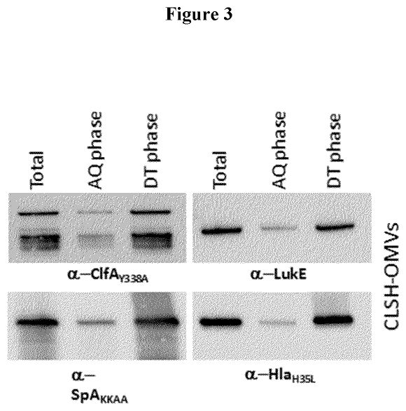

Since the fusion proteins were expressed fused to the lipoprotein leader sequence they are expected to compartmentalize in the OMV membrane. To confirm that, vesicles containing the proteins of interest were solubilized at 4° C. with a 1% water solution of Triton X-114 and subsequently the samples were warmed to 37° C. to partition Triton X-114 into two phases: a detergent-rich hydrophobic phase and a detergent-poor hydrophilic phase. Membrane proteins, including lipoproteins, typically partition selectively into the Triton X-114 hydrophobic phase (Bordier, 1981). Aliquots from both aqueous and organic phases were analysed by Western Blot using antibodies specific for the corresponding S. aureus antigens. As shown in FIG. 3, both the two chimeric proteins are enriched in the hydrophobic phase (DT phase).

CLSH-OMV Immunization Elicits Antigen-Specific Antibody Responses

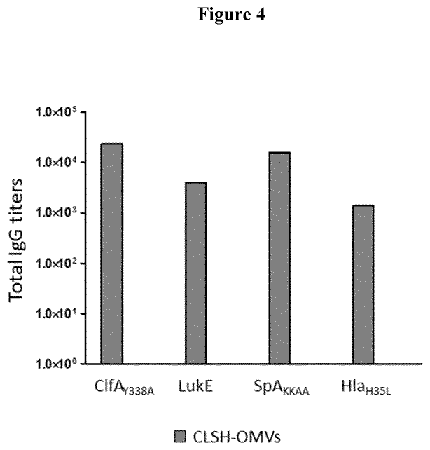

To test whether CLSH-OMVs were capable of inducing specific antibody responses, CD1 mice were immunized three times at two-week intervals with CLSH-OMVs with 2 mg/ml alum hydroxide. Blood samples were collected seven days after third dose (post3) administration and IgGs against ClfA_{Y338A}, LukE, SpA_{KKAA} and Hla_{H35L} were detected by using plates coated in each well with the relative protein. More specifically, coating was carried out by incubating plates overnight at 4° C. with 100 μl of antigen (3 μg/ml). Subsequently, wells were washed three times with PBST (0.05% Tween 20 in PBS, pH 7.4), incubated with 100 μl of 1% BSA in PBS for 1 hour at room temperature and washed again three times with PB ST. Serial dilutions of serum samples in PBST containing 1% BSA were added to the plates, incubated 2 hours at 37° C., and washed three times with PB ST. Then 100 μl/well of 1:2.000 diluted, alkaline phosphatase-conjugated goat anti-mouse IgGs, were added and left for 2 hours at 37° C. After triple PBST wash, bound alkaline phosphatase-conjugated antibodies were detected by adding 100 μl/well of 3 mg/ml para-nitrophenyl-phosphate disodium hexahydrate (Sigma-Aldrich) in 1M diethanolamine buffer (pH 9.8). After 20-minute incubation at room temperature the substrate hydrolysis was analyzed at 405 nm in a microplate spectrophotometer. As shown in FIG. 4, immunization elicited antibodies specific for each of the four S. aureus antigens present in the OMVs. Antibody titers against HlaH35L, SpA_{KKAA} and LukE were similar to what obtained immunizing mice with OMVs engineered with single antigens (Irene C et al (2019) Proc. Natl. Acad. Sci. USA, 116: 21780-21788). Since ClfA_{Y338A}-OMVs were not included in previous studies, no comparative analysis could be done. However, ClfA_{Y338A} appeared to be the most immunogenic antigen among the four present in CLSH-OMVs.

CLSH-OMV Immunization Elicits Antigen-Specific Functional Antibody

We also tested whether the antibodies against ClfA_{Y338A} and HlaH35L could have the capacity of inhibiting the biological function of the corresponding S. aureus ClfA and Hla virulent factors.

The inhibition of the hemolytic activity of Hla was tested by incubating sera from mice immunized with CLSH-OMVs with purified Hla and rabbit erythrocytes. Briefly, mice sera were twofold serially diluted in PBS+0.5% BSA in a 96-well plate with round bottom. Forty μl of each serum dilution is incubated with 10 μl of rHla (Abcam) at a concentration of 2 ng/ml for 20 minutes at room temperature. Fifty μl of water and 50 μl of PBS+0.5% BSA are dispensed in 4 wells as positive (maximal hemolysis) and negative control, respectively. Fifty μl of a solution of 2% rabbit blood erythrocytes are then added to each well and the plate is incubated at 37° C. for 30 minutes. The plate is then centrifuged at 1.000×g at 4° C. for 5 minutes and 70 μl of the supernatants were transferred in a 96-well plate with flat bottom. One-hundred μl of PBS were added in each well and the absorbance was read at 540 nm in a plate reader. Percentage of hemolysis was calculated with respect to the hemolysis given by 50 μl of water (100%).

To follow ClfA inhibition, plates were coated with fibrinogen and the binding of S. aureus Newman strain in the presence or absence of sera from CLSH-OMV-immunized mice was analyzed. Briefly, a 96-well microtiter plate was coated with 10 μg/ml human Fibrinogen (Fg, Sigma #341517) in sterile PBS overnight at 4° C. (100W/well). Microtiter plate wells were then blocked with sterile BSA 5% (p/v) in PBS (100W/well) for 2 hours at 37° C. After blocking, wells were washed 3 times with sterile PBS (100W/well). Immune sera (30 μl, serially diluted in PBS) were pre-incubated separately (in a different 96-well microtiter plate) with 10\hat{ }7 CFUs of S. aureus Newman strain in PBS (100W/well) for 30 minutes at room temperature.

Bacteria plus immune sera were then transferred to the Fg-coated plate wells for 1 hour at 37° C.

Supernatant was removed, adherent cells were washed once with sterile PBS (100 μl/well) and fixed with formaldehyde (FA) 2.5% (v/v) in sterile PBS for 30 minutes at room temperature (1001A/well).

After washing, bacteria were stained with crystal violet (CV, Sigma #V5265) 0.5% (v/v) (1251A/well) for 10 minutes at room temperature, washed once with sterile PBS (150 μl/well) and air-dried. Absorbance was read at 595 nm using a SpectraMax M2 Microplate reader (Molecular Devices). Percentage of inhibition of bacterial binding to Fg was calculated with respect to maximal bacterial binding (bacteria without serum, 100% binding).

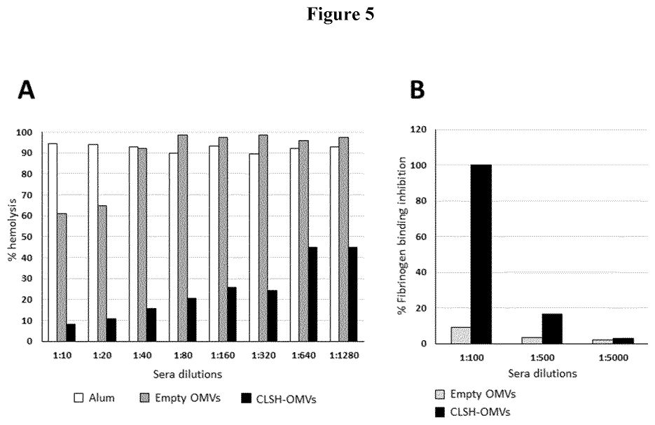

As shown in FIG. 5 the pool of mouse sera from CLSH-OMV-immunized group inhibited the activities of the two virulence factors.

CLSH-OMVs Effectively Protect Mice from the Challenge with Newman Strain

The protective activity of CLSH-OMV immunization in mice challenged with S. aureus Newman strain was evaluated using three different challenge models. The sepsis model, the kidney abscess model and the skin model.

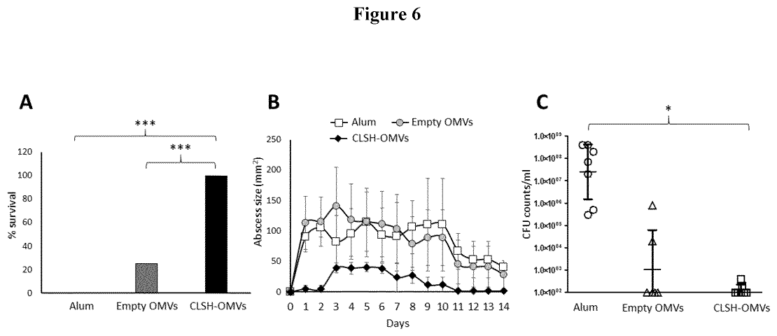

For the sepsis model, mice were immunized three times with 20 μg of OMVs and 14 days after the last immunization the animals received 3×10\hat{ }8 CFUs of bacteria (i.p.). The health status of the animals was followed every day over a period of seven days and survival was evaluated. FIG. 6A reports the data of mice immunized with Alum alone, Empty OMVs and CLSH-OMVs. As shown in the figure, immunization with Empty OMVs conferred a certain level of protection, with 30% of the animals that survived. By contrast, 100% protection was observed in the groups of mice immunized with CLSH-OMVs vaccine. The protective activity of the CLSH-OMVs was also tested in the skin infection model and in the kidney abscess model. As shown in FIG. 6 (B and C), CLSH-OMVs vaccination elicits high protection in both models. In particular, immunized animals challenged with 5×10\hat{ }7 CFUs s.c. developed mild and transient skin abscess. Moreover, using the kidney abscess model according to which 10\hat{ }7 CFUs were given intravenously and bacteria were counted in the kidneys four days after challenge, the CFUs counts in all immunized mice were below the detection limit (10\hat{ }2 CFUs). As previously shown (Irene C et al (2019) Proc. Natl. Acad. Sci. USA, 116: 21780-21788), immunization with Empty OMVs also resulted in a substantial level of protection, confirming the protective role of an innate type of response induced by the OMVs.

Broad Applicability of the Multi-Antigen Engineering of OMVs

To demonstrate the flexibility of the four-antigen engineering of OMVs, different two-antigen chimeras including the S. aureus antigen FhuD2 were created and different couples in which the order of the antigens was modified, were successfully expressed in OMVs. The new combinations of chimeras were shown to induce antigen-specific antibody responses in animals.

1. A method of producing a bacterial outer membrane vesicle (OMV) which comprises:
(i) providing a plurality of polynucleotides, wherein each polynucleotide encodes a fusion product containing (a) at least two different bacterial proteins or (poly)peptides capable of eliciting an immune response in a host, optionally separated by a peptide linker and (b) a leader sequence for secretion at the 5′ end;
(ii) inserting the polynucleotides in expression plasmids, one plasmid for each different polynucleotide, thereby obtaining a plurality of plasmids;
(iii) introducing the plurality of plasmids in a Gram-negative bacterium;
(iv) growing the bacterium under suitable conditions to produce the OMVs.

2. The method of claim 1, wherein the plurality of polynucleotides consists of polynucleotides differing from each other for at least one of the proteins or (poly)peptides in the encoded fusion product.

3. The method of claim 1, wherein the fusion product comprises 2, 3 or 4, different bacterial proteins or (poly)peptides.

4. The method of claim 3, wherein said bacterial proteins or (poly)peptides are Staphylococcus aureus antigens selected from Protein A (SpA) SEQ ID NO:2; Clumping Factor A (ClfA) SEQ ID NO:6; α-hemolysin (Hla) SEQ ID NO:10; and Leukocidin-subunit E (LukE) SEQ ID NO:14; or variants thereof having at least 40%-sequence identity.

5. (canceled)

6. The method of claim 1, wherein the fusion products contain the following antigens, optionally separated by a peptide linker:
(a) SpA_{KKAA} and Hla_{H35L} (SEQ ID NO:16),
and
(b) ClfA_{Y338A} and LukE (SEQ ID NO:18).

7. The method of claim 1, wherein the peptide linker consists of 1 to 20 amino acids.

8. The method of claim 1, wherein the leader sequence for secretion is a leader sequence which promotes the translocation of the fusion proteins into the periplasm of the Gram-negative bacterium, thus allowing the compartmentalization of the fusion proteins in the lumen of the OMV.

9. The method of claim 1, wherein the leader sequence is a lipoprotein leader sequence which promotes the translocation of the fusion proteins into the outer membrane of the Gram-negative bacterium thus allowing the compartmentalization of the fusion proteins as lipidated proteins in the membrane of the OMV.

10. The method of claim 1, wherein the expression plasmid is capable of replication in a Gram-negative bacterium selected from pGEX, pUC19, pALTR, pQE, pLEX and pHAT.

11. The method of claim 10, wherein different plasmids have compatible origins of replication.

12. The method of claim 10, wherein said plasmid comprises the polynucleotide operably linked to a transcription promoter and a translation element.

13. The method of claim 1, wherein the Gram-negative bacterium is E. coli.

14. An isolated bacterial outer membrane vesicle (OMV) carrying in the lumen or in the membrane a plurality of different bacterial proteins or (poly)peptides capable of eliciting an immune response in a host, wherein said proteins or (poly)peptides are fused to each other in groups of two or more, optionally separated by a peptide linker.

15. The OMV of claim 14, wherein said proteins or (poly)peptides capable of eliciting an immune response in a host are bacterial antigens.

16. The OMV of claim 14, wherein said proteins or (poly)peptides consist of 4 different bacterial antigens fused to each other in groups of two, optionally separated by a peptide linker.

17. The OMV of claim 15, wherein said antigens are Staphylococcus aureus antigens selected from Protein A (SpA) SEQ ID NO:2; Clumping Factor A (ClfA) SEQ ID NO:6; α-hemolysin (Hla) SEQ ID NO:10; and Leukocidin-subunit E (LukE) SEQ ID NO:14; or variants thereof having at least 40%, sequence identity.

18. (canceled)

19. The OMV of claim 17, wherein said S. aureus antigens are fused to each other in the following combinations:
(a) SpA_{KKAA} and Hla_{H35L} (SEQ ID NO:16),
and
(b) ClfA_{Y338A} and LukE (SEQ ID NO:18)

20. (canceled)

21. An immunogenic composition containing the OMVs of claim 14.

22. The immunogenic composition of claim 21, which is in the form of a vaccine.

23. (canceled)

24. A method of stimulating an immune response in a subject in need thereof with the OMV of claim 14 or with an immunogenic composition containing the OMV of claim 14, said method comprising
administering to said subject said OMV or said immunogenic composition, wherein said subject is an individual with a disease correlated to or caused by a bacterial infection caused by S. aureus or an individual at risk of developing a bacterial infection.

25. (canceled)


SEQUENCE LISTING (23 sequences)

SEQ ID NO: 1 - DNA, 1551 bp, Staphylococcus aureus
ttgaaaaagaaaaacatttattcaattcgtaaactaggtgtaggtattgcatctgtaactttaggtacattacttatatctggtggcgtaacacctgctgcaaatgctgcgcaacacgatgaagctcaacaaaatgctttttatcaagtcttaaatatgcctaacttaaatgctgatcaacgcaatggttttatccaaagccttaaagatgatccaagccaaagtgctaacgttttaggtgaagctcaaaaacttaatgactctcaagctccaaaagctgatgcgcaacaaaataacttcaacaaagatcaacaaagcgccttctatgaaatcttgaacatgcctaacttaaacgaagcgcaacgtaacggcttcattcaaagtcttaaagacgacccaagccaaagcactaacgttttaggtgaagctaaaaaattaaacgaatctcaagcaccgaaagctgataacaatttcaacaaagaacaacaaaatgctttctatgaaatcttgaatatgcctaacttaaacgaagaacaacgcaatggtttcatccaaagcttaaaagatgacccaagccaaagtgctaacctattgtcagaagctaaaaagttaaatgaatctcaagcaccgaaagcggataacaaattcaacaaagaacaacaaaatgctttctatgaaatcttacatttacctaacttaaacgaagaacaacgcaatggtttcatccaaagcctaaaagatgacccaagccaaagcgctaaccttttagcagaagctaaaaagctaaatgatgctcaagcaccaaaagctgacaacaaattcaacaaagaacaacaaaatgctttctatgaaattttacatttacctaacttaactgaagaacaacgtaacggcttcatccaaagccttaaagacgatccttcagtgagcaaagaaattttagcagaagctaaaaagctaaacgatgctcaagcaccaaaagaggaagacaataacaagcctggcaaagaagacaataacaagcctggcaaagaagacaacaacaagcctggtaaagaagacaacaacaagcctggtaaagaagacaacaacaagcctggcaaagaagacggcaacaagcctggtaaagaagacaacaaaaaacctggtaaagaagatggcaacaagcctggtaaagaagacaacaaaaaacctggtaaagaagacggcaacaagcctggcaaagaagatggcaacaaacctggtaaagaagatggtaacggagtacatgtcgttaaacctggtgatacagtaaatgacattgcaaaagcaaacggcactactgctgacaaaattgctgcagataacaaattagctgataaaaacatgatcaaacctggtcaagaacttgttgttgataagaagcaaccagcaaaccatgcagatgctaacaaagctcaagcattaccagaaactggtgaagaaaatccattcatcggtacaactgtatttggtggattatcattagccttaggtgcagcgttattagctggacgtcgtcgcgaactataa

SEQ ID NO: 2 - protein, 516 aa, Staphylococcus aureus
LKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 3 - DNA, 876 bp, Artificial Sequence
  mutant S. aureus antigen
gcacagcatgatgaagccaaaaaaaacgccttttatcaggttctgaatatgccgaatctgaatgccgatcagcgtaatggttttattcagagcctgaaagcagcaccgagccagagcgcaaatgttctgggtgaagcacagaaactgaatgatagccaggcaccgaaagcagatgccaaacgcaacaattttaacaaagataaaaaaagcgcgttttatgaaatcctgaacatgcctaacctgaatgaagcacagcgcaatggctttatccagtctctgaaagccgcaccgtcacagtctaccaatgtgctgggcgaagcgaaaaaactgaacgaatcccaggctccgaaagccgataataacttcaacaaagagaaaaaaaacgccttttatgaaattctgaatatgccaaatctgaacgaagaacagcgtaacggttttattcagtcactgaaagcggctcctagccagtctgcaaatctgctgtctgaagccaaaaaactgaatgaaagtcaggcacctaaagcggataacaaatttaacaaagagaaaaaaaacgcattttatgaaatcctgcatctgccgaatctgaatgaagaacagcgcaacggctttattcagagtctgaaagccgctccgtcccagagcgccaacctgctggccgaagcaaaaaaactgaatgatgcgcaggctccgaaagcagataacaaatttaacaaagagaaaaaaaacgccttctatgaaattctgcacctgcctaacctgaccgaagaacagcgtaatggttttatccagtccctgaaagcggctcctagcgttagcaaagaaatcctggcagaggccaaaaaactgaacgacgcacaggcacctaaataa

SEQ ID NO: 4 - protein, 291 aa, Artificial Sequence
  mutant S. aureus antigen
AQHDEAKKNAFYQVLNMPNLNADQRNGFIQSLKAAPSQSANVLGEAQKLNDSQAPKADAKRNNFNKDKKSAFYEILNMPNLNEAQRNGFIQSLKAAPSQSTNVLGEAKKLNESQAPKADNNFNKEKKNAFYEILNMPNLNEEQRNGFIQSLKAAPSQSANLLSEAKKLNESQAPKADNKFNKEKKNAFYEILHLPNLNEEQRNGFIQSLKAAPSQSANLLAEAKKLNDAQAPKADNKFNKEKKNAFYEILHLPNLTEEQRNGFIQSLKAAPSVSKEILAEAKKLNDAQAPK

SEQ ID NO: 5 - DNA, 1563 bp, Staphylococcus aureus
agtgaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttctaatgatactaatacagtatcatctgtaaattcacctcaaaattctacaaatgcggaaaatgtttcaacaacgcaagatacttcaactgaagcaacaccttcaaacaatgaatcagctccacagagtacagatgcaagtaataaagatgtagttaatcaagcggttaatacaagtgcgcctagaatgagagcatttagtttagcggcagtagctgcagatgcaccggtagctggcacagatattacgaatcagttgacgaatgtgacagttggtattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgtaaatactaaagatgatgtaaaagcaactttgaccatgcccgcttatattgaccctgaaaatgttaaaaagacaggtaatgtgacattggctactggcataggtagtacaacagcaaacaaaacagtattagtagattatgaaaaatatggtaagttttataacttatctattaaaggtacaattgaccaaatcgataaaacaaataatacgtatcgtcagacaatttatgtcaatccaagtggagataacgttattgcgccggttttaacaggtaatttaaaaccaaatacggatagtaatgcattaatagatcagcaaaatacaagtattaaagtatataaagtagataatgcagctgatttatctgaaagttactttgtgaatccagaaaactttgaggatgtcactaatagtgtgaatattacattcccaaatccaaatcaatataaagtagagtttaatacgcctgatgatcaaattacaacaccgtatatagtagttgttaatggtcatattgatccgaatagcaaaggtgatttagctttacgttcaactttatatgggtataactcgaatataatttggcgctctatgtcatgggacaacgaagtagcatttaataacggatcaggttctggtgacggtatcgataaaccagttgttcctgaacaacctgatgagcctggtgaaattgaaccaattccagagtaa

SEQ ID NO: 6 - protein, 520 aa, Staphylococcus aureus
SENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTSNDTNTVSSVNSPQNSTNAENVSTTQDTSTEATPSNNESAPQSTDASNKDVVNQAVNTSAPRMRAFSLAAVAADAPVAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 7 - DNA, 1569 bp, Artificial Sequence
  mutant S. aureus antigen
tccagcagtgaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttttaatgatactaatacagtatcatctgtaaattcacctcaaaattctacaaatgcggaaaatgtttcaacaacgcaagatacttcaactgaagcaacaccttcaaacaatgaatcagctccacagagtacagatgcaagtaataaagatgtagttaatcaagcggttaatacaagtgcgcctagaatgagagcatttagtttagcggcagtagctgcagatgcaccggcagctggcacagatattacgaatcagttgacgaatgtgacagttggtattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgtaaatactaaagatgatgtaaaagcaactttgaccatgcccgctgctattgaccctgaaaatgttaaaaagacaggtaatgtgacattggctactggcataggtagtacaacagcaaacaaaacagtattagtagattatgaaaaatatggtaagttttataacttatctattaaaggtacaattgaccaaatcgataaaacaaataatacgtatcgtcagacaatttatgtcaatccaagtggagataacgttattgcgccggttttaacaggtaatttaaaaccaaatacggatagtaatgcattaatagatcagcaaaatacaagtattaaagtatataaagtagataatgcagctgatttatctgaaagttactttgtgaatccagaaaactttgaggatgtcactaatagtgtgaatattacattcccaaatccaaatcaatataaagtagagtttaatacgcctgatgatcaaattacaacaccgtatatagtagttgttaatggtcatattgatccgaatagcaaaggtgatttagctttacgttcaactttatatgggtataactcgaatataatttggcgctctatgtcatgggacaacgaagtagcatttaataacggatcaggttctggtgacggtatcgataaaccagttgttcctgaacaacctgatgagcctggtgaaattgaaccaattccagagtaa

SEQ ID NO: 8 - protein, 522 aa, Artificial Sequence
  mutant S. aureus antigen
SSSENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTFNDTNTVSSVNSPQNSTNAENVSTTQDTSTEATPSNNESAPQSTDASNKDVVNQAVNTSAPRMRAFSLAAVAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPE

SEQ ID NO: 9 - DNA, 891 bp, Staphylococcus aureus
atggctagcgcagattctgatattaatattaaaaccggtactacagatattggaagcaatactacagtaaaaacaggtgatttagtcacttatgataaagaaaatggcatgcacaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaattaa

SEQ ID NO: 10 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 11 - DNA, 882 bp, Artificial Sequence
  mutant S. aureus antigen
gcagattctgatattaatattaaaaccggtactacagatattggaagcaatactacagtaaaaacaggtgatttagtcacttatgataaagaaaatggcatgttaaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaattaa

SEQ ID NO: 12 - protein, 293 aa, Artificial Sequence
  mutant S. aureus antigen
ADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 13 - DNA, 852 bp, Staphylococcus aureus
aatactaatattgaaaatattggtgatggtgctgaagtaatcaaacgtacggaggatgtaagtagtaagaaatggggcgttactcaaaatgtccaattcgactttgtaaaagataaaaaatataacaaagacgctttaattgttaaaatgcaaggttttattaattccagaacttcattttcagatgtgaagggtagtggatatgaattaactaaacgaatgatttggccattccaatataatataggactgacgactaaagatccaaatgttagcttaatcaattaccttcctaaaaacaaaatagaaactactgatgttggtcaaacattaggatataacattggaggtaatttccagtcagcaccatctataggtggcaatggctcatttaattattctaaaacaattagttatacccaaaagagttatgtcagtgaagtagacaagcaaaactcaaaatctgttaaatggggtgttaaagcaaacgaatttgttacgcctgatggaaaaaaatctgcgcatgatagatatttattcgtacaaagtccaaatggtccaacaggttcagcaagagaatattttgctcctgataatcaattgccacctttagttcaaagtggctttaatccatcgtttatcactacactatcacatgaaaaaggttcaagtgatacgagtgaatttgaaatttcatatggtagaaacttagatattacatatgcgactttattccctagaactggtatttacgcagaaagaaagcataatgcatttgtaaatagaaactttgtagttagatatgaagttaattggaaaacacacgaaattaaagtgaaaggacataattaa

SEQ ID NO: 14 - protein, 283 aa, Staphylococcus aureus
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 15 - DNA, 1833 bp, Artificial Sequence
  SpA(KKAA) - Hla(H35L) fusion product
atgaaagctactaaactggtactgggcgcggtaatcctgggttctactctgctggcaggttgcgcacagcatgatgaagccaaaaaaaacgccttttatcaggttctgaatatgccgaatctgaatgccgatcagcgtaatggttttattcagagcctgaaagcagcaccgagccagagcgcaaatgttctgggtgaagcacagaaactgaatgatagccaggcaccgaaagcagatgccaaacgcaacaattttaacaaagataaaaaaagcgcgttttatgaaatcctgaacatgcctaacctgaatgaagcacagcgcaatggctttatccagtctctgaaagccgcaccgtcacagtctaccaatgtgctgggcgaagcgaaaaaactgaacgaatcccaggctccgaaagccgataataacttcaacaaagagaaaaaaaacgccttttatgaaattctgaatatgccaaatctgaacgaagaacagcgtaacggttttattcagtcactgaaagcggctcctagccagtctgcaaatctgctgtctgaagccaaaaaactgaatgaaagtcaggcacctaaagcggataacaaatttaacaaagagaaaaaaaacgcattttatgaaatcctgcatctgccgaatctgaatgaagaacagcgcaacggctttattcagagtctgaaagccgctccgtcccagagcgccaacctgctggccgaagcaaaaaaactgaatgatgcgcaggctccgaaagcagataacaaatttaacaaagagaaaaaaaacgccttctatgaaattctgcacctgcctaacctgaccgaagaacagcgtaatggttttatccagtccctgaaagcggctcctagcgttagcaaagaaatcctggcagaggccaaaaaactgaacgacgcacaggcacctaaaggtggtggtggcagcgcagattctgatattaatattaaaaccggtactacagatattggaagcaatactacagtaaaaacaggtgatttagtcacttatgataaagaaaatggcatgttaaaaaaagtattttatagttttatcgatgataaaaatcataataaaaaactgctagttattagaacgaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatatgatagagattcttggaacccggtatatggcaatcaacttttcatgaaaactagaaatggctctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgactaccaattgcactggacttcaacaaattggaaaggtaccaatactaaagataaatggatagatcgttcttcagaaagatataaaatcgattgggaaaaagaagaaatgacaaattaa

SEQ ID NO: 16 - protein, 610 aa, Artificial Sequence
  SpA(KKAA) - Hla(H35L) fusion product
MKATKLVLGAVILGSTLLAGCAQHDEAKKNAFYQVLNMPNLNADQRNGFIQSLKAAPSQSANVLGEAQKLNDSQAPKADAKRNNFNKDKKSAFYEILNMPNLNEAQRNGFIQSLKAAPSQSTNVLGEAKKLNESQAPKADNNFNKEKKNAFYEILNMPNLNEEQRNGFIQSLKAAPSQSANLLSEAKKLNESQAPKADNKFNKEKKNAFYEILHLPNLNEEQRNGFIQSLKAAPSQSANLLAEAKKLNDAQAPKADNKFNKEKKNAFYEILHLPNLTEEQRNGFIQSLKAAPSVSKEILAEAKKLNDAQAPKGGGGSADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 17 - DNA, 2496 bp, Artificial Sequence
  ClfA(Y338A)-LukE fusion product
atgaaagctactaaactggtactgggcgcggtaatcctgggttctactctgctggcaggttgctccagcagtgaaaatagtgttacgcaatctgatagcgcaagtaacgaaagcaaaagtaatgattcaagtagcgttagtgctgcacctaaaacagacgacacaaacgtgagtgatactaaaacatcgtcaaacactaataatggcgaaacgagtgtggcgcaaaatccagcacaacaggaaacgacacaatcatcatcaacaaatgcaactacggaagaaacgccggtaactggtgaagctactactacgacaacgaatcaagctaatacaccggcaacaactcaatcaagcaatacaaatgcggaggaattagtgaatcaaacaagtaatgaaacgacttttaatgatactaatacagtatcatctgtaaattcacctcaaaattctacaaatgcggaaaatgtttcaacaacgcaagatacttcaactgaagcaacaccttcaaacaatgaatcagctccacagagtacagatgcaagtaataaagatgtagttaatcaagcggttaatacaagtgcgcctagaatgagagcatttagtttagcggcagtagctgcagatgcaccggcagctggcacagatattacgaatcagttgacgaatgtgacagttggtattgactctggtacgactgtgtatccgcaccaagcaggttatgtcaaactgaattatggtttttcagtgcctaattctgctgttaaaggtgacacattcaaaataactgtacctaaagaattaaacttaaatggtgtaacttcaactgctaaagtgccaccaattatggctggagatcaagtattggcaaatggtgtaatcgatagtgatggtaatgttatttatacatttacagactatgtaaatactaaagatgatgtaaaagcaactttgaccatgcccgctgctattgaccctgaaaatgttaaaaagacaggtaatgtgacattggctactggcataggtagtacaacagcaaacaaaacagtattagtagattatgaaaaatatggtaagttttataacttatctattaaaggtacaattgaccaaatcgataaaacaaataatacgtatcgtcagacaatttatgtcaatccaagtggagataacgttattgcgccggttttaacaggtaatttaaaaccaaatacggatagtaatgcattaatagatcagcaaaatacaagtattaaagtatataaagtagataatgcagctgatttatctgaaagttactttgtgaatccagaaaactttgaggatgtcactaatagtgtgaatattacattcccaaatccaaatcaatataaagtagagtttaatacgcctgatgatcaaattacaacaccgtatatagtagttgttaatggtcatattgatccgaatagcaaaggtgatttagctttacgttcaactttatatgggtataactcgaatataatttggcgctctatgtcatgggacaacgaagtagcatttaataacggatcaggttctggtgacggtatcgataaaccagttgttcctgaacaacctgatgagcctggtgaaattgaaccaattccagagggtggtggtggcagcaatactaatattgaaaatattggtgatggtgctgaagtaatcaaacgtacggaggatgtaagtagtaagaaatggggcgttactcaaaatgtccaattcgactttgtaaaagataaaaaatataacaaagacgctttaattgttaaaatgcaaggttttattaattccagaacttcattttcagatgtgaagggtagtggatatgaattaactaaacgaatgatttggccattccaatataatataggactgacgactaaagatccaaatgttagcttaatcaattaccttcctaaaaacaaaatagaaactactgatgttggtcaaacattaggatataacattggaggtaatttccagtcagcaccatctataggtggcaatggctcatttaattattctaaaacaattagttatacccaaaagagttatgtcagtgaagtagacaagcaaaactcaaaatctgttaaatggggtgttaaagcaaacgaatttgttacgcctgatggaaaaaaatctgcgcatgatagatatttattcgtacaaagtccaaatggtccaacaggttcagcaagagaatattttgctcctgataatcaattgccacctttagttcaaagtggctttaatccatcgtttatcactacactatcacatgaaaaaggttcaagtgatacgagtgaatttgaaatttcatatggtagaaacttagatattacatatgcgactttattccctagaactggtatttacgcagaaagaaagcataatgcatttgtaaatagaaactttgtagttagatatgaagttaattggaaaacacacgaaattaaagtgaaaggacataattaa

SEQ ID NO: 18 - protein, 831 aa, Artificial Sequence
  ClfA(Y338A)-LukE fusion product
MKATKLVLGAVILGSTLLAGCSSSENSVTQSDSASNESKSNDSSSVSAAPKTDDTNVSDTKTSSNTNNGETSVAQNPAQQETTQSSSTNATTEETPVTGEATTTTTNQANTPATTQSSNTNAEELVNQTSNETTFNDTNTVSSVNSPQNSTNAENVSTTQDTSTEATPSNNESAPQSTDASNKDVVNQAVNTSAPRMRAFSLAAVAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPIPEGGGGSNTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 19 - protein, 23 aa, Mus musculus
MKATKLVLGAVILGSTLLAGCSS

SEQ ID NO: 20 - protein, 15 aa, Artificial Sequence
  synthetic peptide
CGTNTKDKWIDRSSE

SEQ ID NO: 21 - protein, 15 aa, Artificial Sequence
  synthetic peptide
CNEFVTPDGKKSAHD

SEQ ID NO: 22 - protein, 15 aa, Artificial Sequence
  synthetic peptide
CAKKLNDAQAPKADN

SEQ ID NO: 23 - protein, 4 aa, Artificial Sequence
  lipoprotein signal sequence
  VARIANT (2)..(2): Ala or Ser residue
  VARIANT (3)..(3): Gly or Ala residue
LXXC